(19) United States
(12) Patent Application Publication
(10) Pub. No.: US 2007/0122855 A1
(43) Pub. Date: May 31, 2007

(54) METHODS FOR DIAGNOSING HEPATOCELLULAR CARCINOMA

(75) Inventors: Shu-Yuan CHIANG, Taipei (TW); Ming-Fong LU, Taipei (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: TARGETGEN INC., Taipei (TW)

(21) Appl. No.: 11/563,866

(22) Filed: Nov. 28, 2006

Related U.S. Application Data
(60) Provisional application No. 60/740,223, filed on Nov. 28, 2005.

(51) Int. Cl. G01N 33/574

(52) U.S. Cl. 435/7.23

(57) ABSTRACT

Methods are provided for hepatocellular carcinoma (HCC) diagnosis. The methods confirm the occurrence of HCC in a subject suspected of suffering from HCC by determining the level of either IL-6 or IL-10 in a plasma sample obtained from the subject. Also provided are methods for distinguishing HCC from other liver diseases by determining the level of either IL-6 or IL-10 in a plasma sample obtained from a subject suffering from a liver disease.

### CROSS-REFERENCE TO RELATED APPLICATIONS

This application claims the benefit of U.S. Provisional Patent Application No. 60/740,223 filed on Nov. 28, 2005, and the disclosure of which is incorporated herein by reference.

### BACKGROUND OF THE INVENTION

The present invention relates to methods for diagnosing hepatocellular carcinoma (HCC).

HCC is one of the most common malignant tumors in the world, with the global incidence increasing annually. HCC often occurs in patients with chronic liver disease, usually resulting from type B or C virus infection. Although surgical resection is an effective treatment for the disease, the rate of tumor recurrence after resection is high. Occult microscopic intrahepatic or extrahepatic metastasis and background factors of the diseased liver, including active inflammation, had been regarded as significant risk factors for tumor recurrence.

The single most important tumor marker for HCC is α-fetoprotein (AFP). HCC surveillance with serum AFP level and ultrasonography has been recommended for patients with cirrhosis. Although the detection of serum AFP level is well established in the screening and diagnostic procedures for HCC, a major shortcoming is that serum AFP is insensitive to early cancer detection. There are other promising tumor markers, such as des-gamma-carboxy prothrombin, lens culinaris agglutinin-reactive AFP, pancreatitis-associated protein, and insulin-like growth factor-1, but none of these markers has been validated for clinical use.

Human hepatocyte growth factor (HGF) was first described as a hepatotrophic factor in partially hepatectomized rat plasma in the early 1980's and was purified from plasma of a patient with fulminant hepatic failure and from rat platelets in 1986-1987. It has been revealed that HGF is the same protein as scatter factor and tumor cytotoxic factor, and is now known to be a broad-spectrum growth factor which stimulates cell growth not only of hepatocytes but also of many other types of epithelial and endothelial cells. In humans, plasma levels of HGF increase to greater than 10 ng/ml during severe liver disease such as fulminant hepatic failure and decrease rapidly to normal levels when the patients recover from the disease. In less severe liver damage such as occurs in acute hepatitis, levels of HGF in plasma increase to 0.5-1 ng/ml which is approaching the half maximal concentration for the stimulation of DNA synthesis in human hepatocytes in culture. Thus, HGF is believed to be involved in control of liver regeneration. HGF has been shown to link with some human cancers, including HCC.

Interleukin-6 (IL-6) and Interleukin-10 (IL-10) are multifunctional cytokines produced by a range of cells and play a central role in host defense mechanism and modulation of immune response. Recently, it was shown that IL-6 is produced by human epidermal cells and epidermoid carcinoma cell lines, as well as other epithelial tumors, including bladder carcinoma, renal cell carcinoma, and ovarian cancer. Increased serum IL-6 levels have been found in patients with multiple myeloma, renal cell carcinoma, bladder carcinoma, head and neck cancer, ovarian cancer, and cholangiocarcinoma. High serum IL-6 levels had been observed in patients with advanced HCC (Malaguarnera et al., “Role de l'interleukine 6 dans le carcinoma hepatocellulaire,” (1996) Bull Cancer 83:379-384).

IL-10 is a pleiotropic cytokine produced by macrophages, T-helper 2 cells, and B lymphocytes (CD5 subset) and can both stimulate and suppress the immune response. IL-10 has been shown to inhibit various immune functions, such as antigen presentation, cytokine production, macrophage activation, and antigen-specific T-cell proliferation. By interfering with the costimulatory function of antigen-presenting cells (e.g., downregulation of class II MHC expression of monocytes and costimulatory molecule expression of macrophages), IL-10 reduces antigen-specific T-cell proliferation. Recently, it has been proposed that IL-10 plays a key role in the oncogenetic and metastatic ability of neoplasms, and increased levels have been found in the plasma of patients with different histotypes of solid and hematopoietic tumors. Serum IL-10 levels have been observed to be significantly elevated in patients with type C chronic liver disease, and IL-10 may be related to the development of HCC (Kakumu et al., “Serum levels of IL-10, IL-15 and soluble tumor necrosis factor-alpha (TNF-a) receptors in type C chronic liver disease,” (1997) Clin Exp Immunol 109:458-463).

It is still not known whether serum levels of IL-10 and IL-6 are related to the prognosis of patients with relatively early and resectable HCC. The potential role of IL-6, IL-10 and HGF as tumor markers for HCC and their relationship with AFP are not fully clear. There remains a need for an effective method for HCC diagnosis, especially in the early stage of the disease.

### BRIEF SUMMARY OF THE INVENTION

In accordance with one embodiment of the present invention, there is provided a method for confirming the occurrence of HCC in a subject suspected of suffering from HCC, comprising determining the level of IL-6 in the blood of the subject, wherein a level of IL-6 higher than a normal level is diagnostically indicative of HCC.

In accordance with another embodiment of the present invention, there is provided a method for confirming the occurrence of HCC in a subject suspected of suffering from HCC, comprising determining the level of IL-10 in the blood of the subject, wherein a level of IL-10 higher than a normal level is diagnostically indicative of HCC.

In accordance with yet another embodiment of the present invention, there is provided a method for distinguishing HCC from other liver diseases, comprising determining the level of IL-6 in the blood of a subject suffering from a liver disease, wherein a level of IL-6 higher than a normal level is diagnostically indicative of HCC.

In accordance with yet another embodiment of the present invention, there is provided a method for distinguishing HCC from other liver diseases, comprising determining the level of IL-10 in the blood of a subject suffering from a liver disease, wherein a level of IL-10 higher than a normal level is diagnostically indicative of HCC.

### DETAILED DESCRIPTION OF THE INVENTION

Abbreviations:
AFP—α-fetoprotein
ELISA—Enzyme-linked immunosorbent assay
HBV—Hepatitis B virus
HCC—Hepatocellular carcinoma
HCV—Hepatitis C virus
HGF—Hepatocyte growth factor
IL-6—Interleukin-6
IL-10—Interleukin-10

The present invention is directed to novel methods for diagnosing HCC. It has been found that plasma IL-6 and IL-10 levels are frequently elevated in HCC patients but not in patients with benign liver diseases or non-HCC tumors. In contrast, Measurement of HGF level is not useful for differentiating HCC from other conditions. Therefore, IL-6 and IL-10 may be especially helpful in identifying a subset of HCC patients with low AFP level, and may serve as complementary tumor markers that contribute to the differential diagnosis.

In one aspect, the present invention provides methods for confirming the occurrence of HCC in a subject suspected of suffering from HCC, comprising determining the level of either IL-6 or IL-10 in the blood of the subject, wherein a level higher than a normal level is diagnostically indicative of HCC.

As used herein, the term “a level higher than a normal level” refers to a level of a marker that is greater than the level of the marker observed in normal individuals, that is, individuals who are not suffering from HCC. For some markers, no or infinitesimally low levels of the marker may be present normally in an individual's blood. While for others, detectable levels may be present normally in blood. Thus, the term contemplates a level that is significantly above the normal level found in individuals. The term “significantly” refers to statistical significance and generally means a two standard deviation (SD) above normal, or higher, concentration of the marker is present.

The assay method used to determine the level of the tumor markers of the present invention, i.e., IL-6 and IL-10, must be sufficiently sensitive to be able to detect the level of the marker which is present over the concentration range of interest and also must be highly specific. Suitable methods are immunoassay techniques such as sandwich enzyme-linked immunoassays (ELISA), radioimmunoassays (RIA), competitive binding assays, homogeneous assays, and heterogeneous assays. Any suitable immunoassay technique may be utilized, including those which are commercially available. Extensive discussion of the known immunoassay techniques is not required here since such techniques are known to those of skill in the art.

In a preferred embodiment of the present invention, the level of IL-6 or IL-10 in the blood of the subject is determined by an enzyme-linked immunosorbent assay (ELISA).

Generally, a sandwich ELISA comprises the following steps:

1. Preparing a surface to which a known quantity of an antibody is bound;

2. Applying a sample containing the target antigen to the plate;

3. Washing the plate so that unbound antigens are removed;

4. Applying an enzyme-linked antibody which is also specific to the target antigen;

5. Washing the plate so that unbound enzyme-linked antibodies are removed;

6. Applying a chemical which can be converted by the enzyme into a fluorescent signal; and

7. Viewing the result: a fluorescent signal means that the sample contains a detectable amount the target antigen.

The assay devices for the ELISA according to the present invention can be arranged to provide a semiquantitative or a quantitative result. By the term “semiquantitative” is meant the ability to discriminate between a level which is above the elevated marker protein value, and a level which is not above that threshold.

In a preferred embodiment of the present invention, the ELISA for IL-6 or IL-10 is semiquantitative with a sensitivity of 3 pg/mL.

In another aspect, the present invention provides methods for distinguishing HCC from other liver diseases, comprising determining the level of either IL-6 or IL-10 in the blood of a subject suffering from a liver disease, wherein a level higher than a normal level is diagnostically indicative of HCC.

In conformity with previous studies, the results described in the example below indicated increased blood HGF levels (i.e., a blood HGF level higher than normal) in not only HCC patients but also patients suffering from non-HCC tumor and chronic hepatitis. Therefore, measurement of HGF level is not useful for differentiating HCC from other liver diseases. In a preferred embodiment of the present invention, the subject suspected of suffering from HCC has a high plasma level of HGF, preferably higher than 1000 pg/mL.

The method of the present invention is particularly useful in the diagnosis of early-stage HCC, when patients still have a low serum level of AFP. In a preferred embodiment of the present invention, the subject suspected of suffering from HCC has a low serum level of AFP, preferably lower than 20 ng/mL.

Diagnostic methods according to the present invention may involve determining the plasma level of IL-6, IL-10 or both, depends on the discretion of medical professionals.

The present invention is further illustrated by the following example, which is provided for the purpose of demonstration rather than limitation.

### EXAMPLE

Patients and Methods

Patients and Diagnosis

During a 2-year period from October 2002 to September 2004, a total of 128 adults were prospectively enrolled in this study. These patients were categorized into four groups according to different clinical characteristics: group 1 included 29 healthy subjects with normal liver functions; group 2 included 50 patients with known history of chronic hepatitis B or C but without liver tumor; group 3 included 15 patients with benign hepatic hemangioma, 6 patients with liver metastasis from colon or ovary, and 2 patients with cholangiocarcinoma; group 4 included 26 patients with HCC. Serum AFP and plasma IL-6, IL-10, HGF levels were determined in all study subjects. The nature of this study was fully explained to patients according to the standards of Declaration of Helsinki, and this study complies with current ethical guidelines.

Patients were considered to have chronic hepatitis B virus (HBV) infection if patients were seropositive for hepatitis B surface antigen (HBsAg, RIA kits, Abbott Laboratories, North Chicago, Ill., USA) and considered to have hepatitis C virus (HCV) infection if patients were seropositive for antibody against HCV (anti-HCV) by a second-generation enzyme immunoassay (Abbott Laboratories, IL) for at least twice and at least 6 months. Patients with chronic hepatitis B or C infection had normal or mildly elevated (<2 fold increase of upper normal limit) liver enzyme levels in serum. Hepatic hemangioma was diagnosed with typical features in ultrasonography and contrast-enhanced computed tomography scan. Magnetic resonance imaging was used to confirm the presence of hepatic hemangioma in equivocal cases. All patients with a diagnosis of cholangiocarcinoma or solitary metastatic liver cancer had undergone surgical resection for the liver tumor and a confirmed histological diagnosis. Of the 26 HCC patients, 22 had undergone surgical resection and had a pathological proof for HCC; the diagnosis of the remaining 4 patients was established from characteristic imaging findings in ultrasonography, computed tomography scan and hepatic angiography, along with progressively elevated serum AFP levels, according to the diagnostic criteria by the European Association for the Study of the Liver that is also used in Taiwan (Bruix et al., EASL Panel of Experts on HCC. Clinical management of hepatocellular carcinoma: conclusions of the Barcelona-2000 EASL conference. (2001) J Hepatol 35:421-430; and Yeh et al., Hepatic resection for hepatocellular carcinoma in Taiwan. (2002) Eur J Surg Oncol 28:652-6).

Assays for IL-6, IL-10 and HGF

Plasma samples in all study subjects were collected at the time of diagnosis or before surgical resection. Samples were kept at −80° C. and thawed immediately before the determination of the cytokine levels. IL-10 and IL-6 levels were determined using an enzyme-linked immunosorbent assay (ELISA) kit (e-Bioscience, San Diego, Calif., USA), and HGF level was determined using the ELISA kit (DuoSet, R&D Systems, Inc., Minneapolis, Minn., USA) according to the manufacturers' instructions. One hundred microliters of plasma were used in each reaction for all cytokines. The sensitivity of the assay was 3 pg/mL for IL-6 and IL-10, and was 125 pg/mL for HGF. A plasma concentration below these levels was considered non-detectable. All assays were performed independently by laboratory personnel who did not have clinical information.

Statistical Methods

Chi-squared test or Fisher's exact test (two-tailed) was used for categorical data. Mann-Whitney ranked sum test or Kruskal-Wallis one-way ANOVA was used when appropriate for continuous data. Pearson's correlation analysis was used to estimate the correlation between AFP and cytokine levels, or between tumor size and cytokine levels.

Results

Comparison of IL-6, IL-10 and HGF among patients with HCC, non-HCC Tumor, Chronic Hepatitis and Normal Subjects

The details of the comparison among four groups of patients were shown in Table 1. The expression of IL-6 or IL-10 (≧3 pg/mL), or high levels of HGF (>1000 pg/mL) or AFP (>20 ng/mL) was observed in only 0-3% of normal subjects. Among patients with HCC, 46%, 50% and 62% of them had detectable IL-6, IL-10 and AFP>20 ng/mL, respectively, compared to 0-16% of patients in the other three groups that did so (p values all<0.05). For the expression of HGF, although 60% of HCC patients had an HGF level>1000 pg/mL, 52% of patients with chronic hepatitis (p=0.809) and 35% of patients with non-HCC tumor (p=0.154) also had high HGF levels. For IL-6 and IL-10, patients in HCC group had a significantly higher level of HGF compared to normal subjects, patients with chronic hepatitis and non-HCC tumor (p values all<0.05). Patients with HCC had a significantly higher HGF level compared to normal subjects; however, there was no significant difference of the HGF level between HCC and chronic hepatitis groups, or between HCC and non-HCC tumor groups (p values all>0.05).

TABLE 1
Comparison of the detection of IL-6, IL-10 and HGF among
healthy subjects, patients with chronic hepatitis, patients
with non-HCC tumors and patients with HCC tumors

| | Healthy | Chronic | Non-HCC | |
|---|---|---|---|---|
| | subjects | hepatitis | tumor | HCC |
| No. of patients | 29 | 50 | 23 | 26 |
| Age (years)^{a} | 41 ± 10 | 60 ± 15 | 56 ± 14 | 59 ± 13 |
| Male | 45% | 72% | 70% | 69% |
| IL-6^{b} | | | | |
| Detectable | 3% | 4% | 9% | 46% |
| Non-detectable | 967% | 96% | 91% | 54% |
| IL-10^{c} | | | | |
| Detectable | 0 | 2% | 9% | 50% |
| Non-detectable | 100% | 98% | 91% | 50% |
| AFP^{d} | | | | |
| >20 ng/mL | 0 | 16% | 4% | 62% |
| <20 ng/mL | 100% | 84% | 96% | 38% |
| HGF^{e} | | | | |
| >1000 pg/mL | 3% | 52% | 35% | 58% |
| <1000 pg/mL | 97% | 48% | 65% | 42% |

^{a}p = 0.001;
^{b}p < 0.001;
^{c}p < 0.001;
^{d}p < 0.001;
^{e}p < 0.001.
P values indicate statistical difference among 4 groups of subjects.

The sensitivity and specificity of these cytokines to detect HCC were determined in 99 patients with chronic hepatitis (group 2), non-HCC tumor (group 3) and HCC (group 4). The sensitivity of IL-6, IL-10, HGF (>1000 pg/mL) and AFP (>20 ng/mL) was 46%, 50%, 58% and 62% respectively, and the specificity was 95%, 96%, 53% and 88% respectively. The analysis was further stratified according to serum AFP level and shown in Table 2. Among patients with low (<20 ng/mL) AFP level, 40% of 10 HCC patients had detectable IL-6 or IL-10, and the expression of IL-6 or IL-10 was significantly associated with the existence of HCC (p=0.005 and 0.001 respectively). However, there was no significant difference between the expression of HGF and HCC. In contrast, among patients with high (>20 ng/mL) AFP level, there was no significant difference between the expression of all cytokines and HCC.

TABLE 2
Detection of IL-6, IL-10 and HGF in 99 patients with HCC,
chronic hepatitis or non-HCC according to serum AFP level

HCC

| | Yes | No |
|---|---|---|
| AFP <20 ng/mL (n = 74) | | |
| ^{a}IL-6 detectable | 4 | 3 |
| non-detectable | 6 | 61 |
| ^{b}IL-10 detectable | 4 | 1 |
| non-detectable | 6 | 63 |
| ^{c}HGF >1000 pg/mL | 3 | 27 |
| <1000 pg/mL | 7 | 37 |
| AFP >20 ng/mL (n = 25) | | |
| ^{d}IL-6 detectable | 8 | 1 |
| non-detectable | 8 | 8 |
| ^{e}IL-10 detectable | 9 | 2 |
| non-detectable | 7 | 7 |
| ^{f}HGF >1000 pg/mL | 12 | 7 |
| <1000 pg/mL | 4 | 2 |

^{a}p = 0.005;
^{b}p = 0.001;
^{c}p = 0.515;
^{d}p = 0.088;
^{e}p = 0.208;
^{f}p = 1.0

Correlation Between IL-6, IL-10, HGF and AFP Level in HCC Patients

Of the 26 HCC patients, there was a significant association between AFP and IL-6 level (r=0.509, p=0.008), between AFP and IL-10 level (r=0.487, p=0.012), and between IL-6 and IL-10 level (r=0.834, p<0.001) in logarithmic scale.

Association of IL-6, IL-10 and AFP Level with Tumor Size of HCC

The association between tumor sizes and the cytokine levels in HCC patients were analyzed and shown in Table 3. Patients with large (>5 cm) HCC more often had a higher (>20 ng/mL) AFP level and IL-6 and IL-10 expression (p values all<0.05).

TABLE 3
Relation between tumor size and AFP, IL-6
and IL-10 levels in 26 HCC patients

| | Tumor size >5 cm | Tumor size ≦ 5 cm |
|---|---|---|
| AFP^{a} | | |
| >20 ng/mL | 12 | 4 |
| <20 ng/mL | 2 | 8 |
| IL-6^{b} | | |
| Detectable | 10 | 2 |
| Non-detectable | 4 | 10 |
| IL-10^{c} | | |
| Detectable | 10 | 3 |
| Non-detectable | 4 | 9 |

^{a}p = 0.014;
^{b}p = 0.008;
^{c}p = 0.047

Discussion

HGF and HCC

In the above study, we have determined whether the plasma levels of HGF, IL-6 and IL-10 are increased in patients with different diagnostic categories of liver disease. Interestingly, although increased HGF levels in serum or tissue have been reported in patients with HCC (Guirouilh et al., “Expression of hepatocyte growth factor in human hepatocellular carcinoma,” (2001) J Hepatol 34:78-83; and Yamagamim et al., “Serum concentrations of human hepatocyte growth factor is a useful indicator for predicting the occurrence of hepatocellular carcinomas in C-viral chronic liver diseases,” (2002) Cancer 95:824-834), in our series there was no significant difference of the HGF levels in patients with HCC, non-HCC tumor and chronic hepatitis group. However, patients in these three groups did have a higher HGF level compared to normal subjects (Table 1). It is well known that HCC is frequently associated with chronic hepatitis B or C infection and liver cirrhosis. A major function of a tumor marker is that the marker should be able to differentiate patients who truly have cancer from those who do not. Our results show that HGF levels may also be elevated in liver disease-associated conditions other than HCC, suggesting measurement of HGF level is not helpful in differentiating HCC from other non-HCC conditions.

IL-6, IL-10 and HCC

Our previous study indicated that both IL-6 and IL-10 levels were elevated in HCC patients compared to normal controls, and the high levels would invariably decrease after surgical resection. In addition, a high IL-10 level predicted a poor disease-free survival in patients undergoing curative surgery (Chau et al., “Serum interleukin-10 but not interleukin-6 is related to clinical outcome in patients with resectable hepatocellular carcinoma,” (2000) Ann Surg 231:552-558). In this study, we found that both IL-6 and IL-10 expression were more often higher in HCC patients compared to patients in other disease categories. Interestingly, among patients with low (<20 ng/mL) AFP level and different liver disease categories, our data indicated that the expression of IL-6 and IL-10, but not HGF, were closely associated the existence of HCC. It has been estimated that up to 75% of patients with small HCC and 20% of patients with large HCC may have normal serum AFP level that could escape from HCC surveillance. In the current study, 39% (10/26) of HCC patients had serum AFP level<20 ng/mL. Since the diagnosis of HCC could be very difficult in high-risk patients when these patients had low AFP level or tumors mimicking HCC, our results suggest that IL-6 and IL-10 are helpful to identify a subset of HCC patients with low AFP level, and may serve as complementary tumor markers and contribute to differential diagnosis in these patients.

Association of IL-6, IL-10 and AFP in Relation to Tumor Size

Our results also suggested that the expression between IL-6, IL-10 and AFP in HCC patients was intimately associated. Further evidence supporting the role of IL-6 and IL-10 as tumor markers for HCC is that patients with large HCC significantly more often had detectable IL-6 and IL-10, whereas tumor size has been considered the key factor associated with AFP production as well as overall survival in patients undergoing resection or liver transplantation. In addition, IL-6, IL-10 and AFP shared fairly similar profiles of sensitivity and specificity in terms of detecting HCC. Taken altogether, our data indicated IL-6 and IL-10, in addition to AFP, may also be useful tumor markers for HCC.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

The disclosures of the citations mentioned above are all incorporated herein by reference.

We claim:

1. A method for confirming occurrence of hepatocellular carcinoma (HCC) in a subject suspected of suffering from HCC, the method comprising determining the level of interleukin-6 (IL-6) in the blood of said subject, wherein a level of IL-6 higher than a normal level is diagnostically indicative of HCC.

2. The method according to claim 1, wherein a level of IL-6 equal to or higher than 3 pg/mL, determined by an enzyme-linked immunosorbent assay (ELISA) utilizing a plasma sample obtained from said subject, is diagnostically indicative of HCC.

3. The method according to claim 1, wherein the subject has a low serum level of α-fetoprotein (AFP).

4. The method according to claim 3, wherein the serum level of AFP is lower than 20 ng/mL.

5. The method according to claim 1, wherein the subject has a high plasma level of hepatocyte growth factor (HGF).

6. The method according to claim 5, wherein the plasma level of HGF is higher than 1000 pg/mL.

7. The method according to claim 1, further comprising the step of determining the level of interleukin-10 (IL-10) in the blood of said subject, wherein a level of IL-10 higher than a normal level is diagnostically indicative of HCC.

8. The method according claim 7, wherein a level of IL-10 equal to or higher than 3 pg/mL, determined by an enzyme-linked immunosorbent assay (ELISA) utilizing a plasma sample obtained from said subject, is diagnostically indicative of HCC.

9. A method for confirming occurrence of hepatocellular carcinoma (HCC) in a subject suspected of suffering from HCC, the method comprising determining the level of interleukin-10 (IL-10) in the blood of said subject, wherein a level of IL-10 higher than a normal level is diagnostically indicative of HCC.

10. The method according claim 9, wherein a level of IL-10 equal to or higher than 3 pg/mL determined by an enzyme-linked immunosorbent assay (ELISA) utilizing a plasma sample obtained from said subject, is diagnostically indicative of HCC.

11. The method according to claim 9, wherein the subject has a low serum level of α-fetoprotein (AFP).

12. The method according to claim 11, wherein the serum level of AFP is lower than 20 ng/mL.

13. The method according to claim 9, wherein the subject has a high plasma level of hepatocyte growth factor (HGF).

14. The method according to claim 13, wherein the plasma level of HGF is higher than 1000 pg/mL.

15. The method according to claim 9, further comprising the step of determining the level of interleukin-6 (IL-6) in the blood of said subject, wherein a level of IL-6 higher than a normal level is diagnostically indicative of HCC.

16. The method according claim 15, wherein a level of IL-6 equal to or higher than 3 pg/mL, determined by an enzyme-linked immunosorbent assay (ELISA) utilizing a plasma sample obtained from said subject, is diagnostically indicative of HCC.

17. A method for distinguishing hepatocellular carcinoma (HCC) from other liver diseases, the method comprising determining the level of interleukin-6 (IL-6) in the blood of a subject suffering from a liver disease, wherein a level of IL-6 higher than a normal level is diagnostically indicative of HCC.

18. The method according to claim 17, wherein the liver disease other than HCC is selected from the group consisting of chronic hepatitis B, chronic hepatitis C, hepatic hemangioma, cholangiocarcinoma, and metastatic liver cancer.

19. The method according to claim 17, wherein a level of IL-6 equal to or higher than 3 pg/mL, determined by an enzyme-linked immunosorbent assay (ELISA) utilizing a plasma sample obtained from said subject, is diagnostically indicative of HCC.

20. The method according to claim 17, further comprising the step of determining the level of interleukin-10 (IL-10) in the blood of said subject, wherein a level of IL-10 higher than a normal level is diagnostically indicative of HCC.

21. The method according claim 17, wherein a level of IL-10 equal to or higher than 3 pg/mL, determined by an enzyme-linked immunosorbent assay (ELISA) utilizing a plasma sample obtained from said subject, is diagnostically indicative of HCC.

22. A method for distinguishing hepatocellular carcinoma (HCC) from other liver diseases, the method comprising determining the level of interleukin-10 (IL-10) in the blood of a subject suffering from a liver disease, wherein a level of IL-10 higher than a normal level is diagnostically indicative of HCC.

23. The method according to claim 22, wherein the liver disease other than HCC is selected from the group consisting of chronic hepatitis B, chronic hepatitis C, hepatic hemangioma, cholangiocarcinoma, and metastatic liver cancer.

24. The method according to claim 22, wherein a level of IL-10 equal to or higher than 3 pg/mL, determined by an enzyme-linked immunosorbent assay (ELISA) utilizing a plasma sample obtained from said subject, is diagnostically indicative of HCC.

25. The method according to claim 22, further comprising the step of determining the level of interleukin-6 (IL-6) in the blood of said subject, wherein a level of IL-6 higher than a normal level is diagnostically indicative of HCC.

26. The method according claim 25, wherein a level of IL-6 equal to or higher than 3 pg/mL, determined by an enzyme-linked immunosorbent assay (ELISA) utilizing a plasma sample obtained from said subject, is diagnostically indicative of HCC.
